(19) United States
(12) Patent Application Publication
(10) Pub. No.: US 2005/0154191 A1
(43) Pub. Date: Jul. 14, 2005

(54) Cat immunisation vectors

(76) Inventor: Michael Steward, Essex (GB)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/505,546

(22) PCT Filed: Feb. 21, 2003
(86) PCT No.: PCT/GB03/00740

(30) Foreign Application Priority Data
Feb. 22, 2002 (GB) 0204154.9

(51) Int. Cl. C07K 14/705; C07H 21/04

(52) U.S. Cl. 530/350; 536/23.5; 435/69.1; 435/320.1; 435/325

(57) ABSTRACT

Cat C3d polypeptide, fragments thereof, and nucleic acid sequences encoding the polypeptide and fragments are described. Genetic constructs encoding such polypeptides can be used to enhance the immunogenicity of antigens in cats. Variant nucleic acid sequences encoding cat C3d polypeptides, when used to express tandem arrays of the polypeptide show enhanced stability, leading to high level expression in eukaryotic and prokaryotic cell expression systems. When incorporated into a DNA immunization vector or a recombinant live organism vaccine, the variant sequences have reduced risk of undergoing homologous recombination with genomic DNA compared to wild-type sequences, thus reducing the risk of potentially damaging integration events.

This invention relates to cat C3d polypeptide, or fragments thereof, and to nucleic acid sequences encoding such polypeptide or fragments. The invention also relates to genetic constructs comprising genetic sequences encoding cat polypeptides designed to enhance the immunogenicity of antigens in cats and to methods for the generation of such constructs.

The invention further relates to the use of variant nucleic acid sequences to encode cat C3d polypeptides which, when used to express tandem arrays of the polypeptide show enhanced stability, leading to high level expression in eukaryotic and prokaryotic cell expression systems. When incorporated into a DNA immunization vector or a recombinant live organism vaccine, such sequences have reduced risk of undergoing homologous recombination with genomic DNA compared to wild-type sequences, thus reducing the risk of potentially damaging integration events.

A cat polypeptide linked to an antigen,. or nucleic acid encoding the same may be administered as part of a prophylactic or therapeutic vaccine formulation to a cat (the host species), or administered with the intention of raising specific antibodies to the antigen in the host species. Such antigens may be derived from any organism including the host species. The cat polypeptides comprise or consist of tandem arrays of a polypeptide which occurs naturally in the host species and which has immunostimulatory properties. Examples of such polypeptides include polypeptides derived from the complement system, as described below. Such tandem arrays, when linked to an antigen may enhance humoral responses to the antigen by several orders of magnitude.

A number of naturally occurring immune modulators, such as cytokines, have been proposed for inclusion into DNA immunization vectors to be expressed concurrently with the antigen. The use of naked DNA as an immunogen has raised concerns about the potential for its integration into the genome of the host species and the possibility of insertional mutagenesis resulting in the inactivation of tumor suppressor genes or the activation of oncogenes. Such concerns apply equally to recombinant live organisms used as vaccines, many of which undergo rounds of self-replication in the host species. Although the studies have shown integration to be a low frequency occurrence with plasmids containing sequences unrelated to the host species the inclusion of genes derived from the genome of the host species increases this risk significantly.

The complement system consists of a set of serum proteins that are important in the response of the immune system to foreign antigens. The complement system becomes activated when its primary components are cleaved and the products, alone or with other proteins, activate additional complement proteins resulting in a proteolytic cascade. Activation of the complement system leads to a variety of responses including increased vascular permeability, chemotaxis of phagocytic cells, activation of inflammatory cells, opsonisation of foreign particles, direct killing of cells and tissue damage. Activation of the complement system may be triggered by antigen-antibody complexes (the classical pathway) or a normal slow activation may be amplified in the presence of cell walls of invading organisms such as bacteria and viruses (the alternative pathway).

The complement system interacts with the cellular immune system through a specific pathway involving C3, a protein central to both classical and alternative pathways. The proteolytic activation of C3 gives rise to a large fragment (C3b) and exposes a chemically reactive internal thiolester linkage which can react covalently with external nucleophiles such as the celi surface proteins of invading organisms or foreign cells. As a result, the potential antigen is ‘tagged’ with C3b and remains attached to that protein as it undergoes further proteolysis to iC3b and C3d, g. The latter fragments are, respectively, ligands for the complement receptors CR3 and CR2. Thus the labelling of antigen by C3b can result in a targeting mechanism for cells of the immune system bearing these receptors.

That such targeting is important for augmentation of the immune response is first shown by experiments in which mice were depleted of circulating C3 and then challenged with an antigen (sheep erythrocytes). Removal of C3 reduced the antibody response to this antigen. The role of C3 was confirmed by studies in animals genetically deficient in either C3 or the upstream components of the complement cascade which generate C3b, i.e. C2 and C4. More recently, it has been shown that linear conjugation of a model antigen with more than two copies of the murine C3d fragment sequence resulted in a very large (1000-10000-fold) increase in antibody response in mice compared with unmodified antigen controls. The increase could be produced without the use of conventional adjuvants such as Freund's complete adjuvant. The mechanism of this remarkable effect was demonstrated to be high-affinity binding of the multivalent C3d construct to CR2 on B-cells, followed by co-ligation of CR2 with another B-cell membrane protein, CD19, and with membrane-bound immunoglobulin to generate a signal to the B-cell nucleus.

In the experiments of Dempsey et al, (1996) the unmodified antigen control and linear fusions with one or two C3d domains were prepared by transfection of the appropriate coding plasmids into L cells followed by the selection of high-expressing clones. The most immunogenic construct, that with three C3d units, had to be expressed transiently in COS cells and this procedure gave a very poor yield of the fusion protein. In part, the low yield could be attributed to the generation of species containing the antigen but with lower molecular weights, corresponding to fewer than three C3d units. It was unclear from the published work of Dempsey et al whether the latter molecules originated by proteolysis of the three- C3d construct or whether they were due to a recombination event in vivo.

Using another expression system but the same C3d constructs as Dempsey et al, we obtained evidence that the generation of molecules with <3 C3d units from DNA encoding 3× C3d repeats is due to loss of one or more C3d units by homologous recombination and not due to post-translational processing (see WO99/35260) and described methods for the generation and selection of stable variant genes resistant to homologous recombination.

The present invention is defined in the appended independent claims. Preferred features of the invention are specified in the subclaims.

This invention may be used in any context where a nucleic acid sequence is included in a medicament where the sequence of the nucleic acid is homologous to a sequence in the genome of the recipient human or animal host. These may be used in the context of gene therapy, therapeutic or prophylactic vaccination or other therapeutic strategies in which nucleic acid forms part of the medicament. It is particularly useful for, but is not restricted to, DNA immunization vectors encoding proteins with immunopotentiating properties derived from the complement system.

Preferred embodiments of this invention relate specifically to an immunostimulatory component of the complement system, and the use of cat components in veterinary vaccines or to raise antibodies in non-human vertebrate species.

The present invention provides:
1. Novel C3d cDNA sequence from cat.
3. High-level expression of oligomers of cat C3d in prokaryotic and eukaryotic systems and maintenance of stable recombinant expression vector stocks.

There is also disclosed a process for preparing an oligomeric polypeptide in vitro or in vivo comprising: constructing an expression vector, which may be a DNA vector or a recombinant live organism encoding the oligomeric polypeptide; introducing the expression vector into a recombinant host cell in vitro or a host organism in vivo; and culturing the recombinant host cell or host organism under conditions for expression of the polypeptide.

The process may further comprise amplifying cat nucleic acid encoding a C3d polypeptide from tissue derived from a cat. The process may further comprise recovering the polypeptide.

Alos provided is a process for preparing a nucleic acid encoding a C3d polypeptide which comprises:
amplifying cat nucleic acid encoding a C3d polypeptide from tissue derived from a cat;
preparing a replicable expression vector from the amplified nucleic acid which encodes the cat C3d polypeptide;
transforming a host cell with the vector;
culturing the transformed host cell under conditions for replication of the expression vector; and recovering the expression vector in a form suitable for DNA immunization.

There is also provided a linear DNA concatameter encoding the oligomeric polypeptide.

Processes of the invention may be performed using conventional recombinant techniques such as described in Sambrook et al., Molecular Cloning : A laboratory manual 2nd Edition. Cold Spring Harbor Laboratory Press (1989) and DNA Cloning vols I, II and III (D. M. Glover ed., IRL Press Ltd).

There is also provided a process for preparing the linear DNA concatamer by condensing appropriate mono-, di- or oligomeric nucleotide units.

The preparation may be carried out chemically, enzymatically, or by a combination of the two methods, in vitro or in vivo as appropriate. Thus, the linear DNA concatamer may be prepared by the enzymatic ligation of appropriate DNA fragments, by conventional methods such as those described by D. M. Roberts et al., in Biochemistry 1985, 24, 5090-5098.

The DNA fragments may be obtained by digestion of DNA containing the required sequences of nucleotides with appropriate restriction enzymes, by chemical synthesis, by enzymatic polymerisation, or by a combination of these methods.

Digestion with restriction enzymes may be performed in an appropriate buffer at a temperature of 20°-70° C., generally in a volume of 50 μl or less with 0.1-10 μg DNA.

Enzymatic polymerisation of DNA may be carried out in vitro using a DNA polymerase such as DNA polymerase 1 (Klenow fragment) in an appropriate buffer containing the nucleoside triphosphates DATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer at a temperature of 4° C. to 37° C., generally in a volume of 50 μl or less.

The chemical synthesis of the linear DNA concatamer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982). Preferably an automated DNA synthesiser (for example, Applied Biosystems 381A Synthesiser) is employed.

The linear DNA concatamer is preferably prepared by ligating two or more DNA molecules which together comprise a DNA sequence encoding the oligomeric polypeptide. The DNA molecules may be obtained by digestion with suitable restriction enzymes of vectors carrying the required coding sequences.

The precise structure of the DNA molecules and the way in which they are obtained depends upon the structure of the desired product. A linear DNA concatamer encoding the oligomeric polypeptide may be constructed using a variety of methods including chemical synthesis of DNA oligonucleotides, enzymatic polymerisation, restriction enzyme digestion and ligation.

Expression of the oligomeric polypeptide encoded by the linear DNA concatamer in a recombinant host cell or in vivo by a recipient of a DNA immunisation vector may be carried out by means of a replicable expression vector capable, in the host cell or in vivo, of expressing the polypeptide from the DNA polymer.

The replicable expression vector may be prepared by cleaving a vector compatible with the host cell to provide a linear DNA segment having an intact replicon, and combining said linear segment with one or more DNA molecules which, together with said linear segment, encode the polypeptide, under ligating conditions.

Ligation of the linear segment and more than one DNA molecule may be carried out simultaneously or sequentially as desired. Thus, the linear DNA concatamer may be preformed or formed during the construction of the vector, as desired.

The choice of vector will be determined in part by the host cell, which may be prokaryotic, such as E. coli, mammalian, such as mouse C127, mouse myeloma, Chinese hamster ovary, or other eukaryotic (fungi e.g. filamentous fungi or unicellular yeast or an insect cell such as Drosophila or Spodoptera). The host cell may also be in a transgenic. animal or a human or animal recipient of a DNA immunization vector. Suitable vectors include plasmids, bacteriophages, cosmids and recombinant viruses derived from, for example, baculoviruses, vaccinia, adenovirus and herpesvirus.

The linear DNA concatamer may be assembled into vectors designed for isolation of stable transformed mammalian cell lines expressing the fragment e.g. bovine papillomavirus vectors in mouse C127 cells, or amplified vectors in Chinese hamster ovary cells.

The preparation of the replicable expression vector may be carried out conventionally with appropriate enzymes for restriction, polymerisation and ligation of the DNA, by procedures described in, for example, Sambrook et al., cited above. Polymerisation and ligation may be performed as described above for the preparation of the linear DNA concatamer. Digestion with restriction enzymes may be performed in an appropriate buffer at a temperature of 20°-70° C., generally in a volume of 50 μl or less with 0.1-10 μg DNA.

A recombinant host cell may be prepared, in accordance with the invention, by transforming a host cell with. a replicable expression vector of the invention under transforming conditions. Suitable transforming conditions are conventional and are described in, for example, Sambrook et al., cited above, or “DNA Cloning” Vol. II, D. M. Glover ed., IRL Press Ltd, 1985.

The choice of transforming conditions is determined by the host cell. Thus, a bacterial host such as E. coli, may be treated with a solution of CaCl₂ or with a solution comprising a mixture of RbCl, MnCl₂, potassium acetate and glycerol, and then with 3-[N-morpholino]-propane-sulphonic acid, RbCl and glycerol or by electroporation as for example described by Bio-Rad Laboratories, Richmond, Calif., USA, manufacturers of an electroporator. Eukaryotic cells in culture may be transformed by calcium co-precipitation of the vector DNA onto the cells or by using cationic liposomes.

DNA immunization vectors may be administered as naked DNA or contained within a viral particle by injection or by other means of delivery including aqueous or non-aqueous formulations via transdermal or mucosal routes.

The invention also provides a host cell transformed with a replicable expression vector of the invention.

Culturing the transformed host cell under conditions for expression of the linear DNA concatamer may be carried out conventionally, as described in, for example, Sambrook et al., and “DNA Cloning” cited above. Thus, preferably the cell is supplied with nutrient and cultured at a temperature below 45° C.

An oligomeric polypeptide of the invention may be recovered by conventional methods. Thus, where the host cell is bacterial such as E. coli and the oligomeric polypeptide is expressed intracellularly, it may be lysed physically, chemically or enzymatically and the oligomeric polypeptide isolated from the resulting lysate. Where the host cell is eukaryotic, the oligomeric polypeptide may be isolated from the nutrient medium. Where the host cell is in a transgenic animal the polypeptide may be recovered from the natural secretory pathways (e.g. where the polypeptide is secreted in the milk of a female transgenic animal). Where the host cell is in a human or animal recipient of a DNA immunization vector or gene therapy vector the oligomeric polypeptide would not normally be recovered, but may be detected in tissues for the purpose of evaluating the utility of the delivery system.

WO99/35260 describes methods for purification and refolding (where required) of protein products expressed in prokaryotic and eukaryotic systems and its contents are incorporated herein by reference.

Nucleic acid of the invention may encode an additional cysteine residue which can be expressed at the carboxy-terminus or other location within a polypeptide of the invention. The utility and post-translational modification of the carboxy-terminal cysteine is described in WO99/35260.

The use of insect cells infected with recombinant baculovirus encoding the oligomeric polypeptide is a preferred general method for preparing complex proteins, particularly an oligomeric polypeptide of the invention encoding C3d oligomers or fusions of the C3d oligomers with an antigen.

The use of DNA immunization vectors or recombinant live organisms is an alternative general method for delivery of an oligomeric polypeptide encoding C3d oligomers fused to antigen in vivo as an immunogen for prophylactic or therapeutic purposes.

### GENERAL METHODS USED IN EXAMPLES

(i) DNA Cleavage

Cleavage of DNA by restriction endonucleases was carried out according to the manufacturer's instructions using supplied buffers (New England Biolabs (U.K.) Ltd., Herts. or Promega Ltd., Hants, UK). Double digests were carried out simultaneously if the buffer conditions were suitable for both enzymes. Otherwise double digests were carried out sequentially where the enzyme requiring the lowest salt condition was added first to the digest. Once the digest was complete the salt concentration was altered and the second enzyme added.

(ii) DNA Ligation

Ligations were carried out using T4 DNA ligase purchased from Promega or New England Biolabs as described in Sambrook et al, (1989) Molecular Cloning: A Laboratory Manual 2nd Edition, Cold Spring Harbor Laboratory Press.

(iii) Plasmid Isolation

Plasmids were isolated using Wizard™ Plus Minipreps (Promega) or Qiex mini or midi kits and Qiagen Plasmid Maxi kit (QIAGEN, Surrey) according to the manufacturer's instructions.

(iv) DNA Fragment Isolation

DNA fragments were excised from agarose gels and DNA extracted using the QIAEX gel extraction kit or Qiaquick (QIAGEN, Surrey, UK), or GeneClean, or GeneClean Spin Kit or MERmaid Kit, or MERmaid Spin Kit (Bio 101 Inc, CA. USA) gel extraction kits according to the manufacturer's instructions.

(v) Introduction of DNA into E. coli

Plasmids were transformed into competent E. coli BL21(DE3) or XL1-blue strains (Studier and Moffat, (1986), J. Mol. Biol .189:113). The E. coli strains were purchased as a frozen competent cultures from Stratagene (Cambridge, UK).

(vi) DNA Sequencing

The sequences were analysed by a Perkin Elmer ABI Prism 373 DNA Sequencer. This is an electrophoretic technique using 36 cm×0.2 mM 4% acrylamide gels, the fluorescently labelled DNA fragments being detected by a charge coupled device camera according to the manufacturer's instructions.

(vii) Production of Oligonucleotides and Synthetic Genes

Oligonucleotides and synthetic genes were purchased from Cruachem, Glasgow, UK or from Sigma-Genosys, Cambridge, UK.

(viii) Generation of Baculovirus Vectors

Plasmids described in this invention having the prefix pBP or pBAC are used to generate baculovirus vectors and express the encoded recombinant polypeptides by the following methods (Sections (viii) to (x))

Purified plasmid DNA was used to generate recombinant baculoviruses using the kits ‘The BacPak Baculovirus Expression System’ (Clontech, CA, USA) or ‘BacVector 3000’ (Invitrogen) according to the manufacturers' protocols. The insect cell line Sf9 (ATCC) was grown in Sf900II medium (Gibco) at certain times supplemented with foetal calf serum (Gibco, Paisley, UK). Cells were transfected with the linearised baculovirus DNA (supplied in the kits) and the purified plasmid. Plaque assays (see method below) were carried out on culture supernatants and a series of ten-fold dilutions thereof to allow isolation of single plaques. Plaques were picked using glass Pasteur pipettes and transferred into 0.5 ml aliquots of growth medium. This is the primary seed stock.

(ix) Plaque Assay of Baculoviruses

1×10⁶ Sf9 cells were seeded as monolayer cultures in 30 mm plates and left to attach for at least 30 minutes. The medium was poured off and virus inoculum in 100 μl growth medium was dripped onto the surface of the monolayer. The plates were incubated for 30 minutes at room temperature, occasionally tilting the plates to prevent the monolayer from drying out. The monolayer was overlaid with a mixture of 1 ml growth medium and 3% (w/v) “Seaplaque” agarose (FMC, ME) warmed to 37° C. and gently swirled to mix in the inoculum. Once set a liquid overlay of lml growth medium was applied. The plates were incubated in a humid environment for 3-5 days.

Visualisation of plaques was achieved by addition to the liquid overlay 1 ml phosphate buffered saline (PBS) containing neutral red solution at 0.1% (w/v) from a stock solution of 1% (w/v) (Sigma, Dorset, UK). Plaques were visible as circular regions devoid of stain up to 3 mm in diameter.

(x) Scale-up of Baculovirus Vectors and Protein Expression

200 μl of the primary seed stock was used to infect 1×10⁶ Sf9 monolayer cell cultures in 30 mm plates. The seed stock was dripped onto the monolayer and incubated for 20 minutes at room temperature, and then overlaid with 1 ml growth medium. The plates were incubated at 27° C. in a humid environment for 3-5 days. The supernatant from these cultures is Passage 1 virus stock. The virus titre was determined by plaque assay and further scale up was achieved by infection of monolayer cultures or suspension cultures at a multiplicity of infection (moi) of 0.1. Virus stocks were passaged a maximum of six times to minimise the emergence of defective virus.

Expression of recombinant proteins was achieved by infection of monolayer or suspension cultures in growth medium with or without foetal calf serum (FCS) . Where FCS was omitted cells conditioned to growth in the absence of FCS were used. Virus stocks between passage 1 and 6 were used to infect cultures at a moi of >5 per cell. Typically, infected cultures were harvested 72 hours post infection and recombinant proteins isolated either from the supernatants or the cells.

(xiv) Protein Purification

A number of standard chromatographic techniques can be used to isolate the C3d-containing proteins, e.g. such methods as ion-exchange and hydrophobic interaction matrixes chromatography utilising the appropriate buffer systems and gradient to purify the target proteins. The properties of the C3d containing fusion polypeptides will vary depending on the nature of the fusion protein. Examples of methods employed are described in WO99/35260.

(xv) Sodium Dodecyl Sulphate Polyacrylamide Gel Electrophoresis (SDS-PAGE)

SDS-PAGE was carried out generally using the Novex system (Novex GmbH, Heidleburg) according to the manufacturer's instructions. Pre-packed gels of Tris/glycine a 4-20% acrylamide gradient were usually used. Samples for electrophoresis, including protein molecular weight standards (for example LMW Kit, Pharmacia, Sweden or Novex Mark 12, Novex, Germany) were usually diluted in 1% (w/v) SDS—containing buffer (with or without 5% (v/v) 2-mercaptoethanol), and left at room temperature for 5 to 30 min before application to the gel.

(xvi) Immunoblotting

(a) Dot Blot

Immobilon membranes (Millipore, Middlesex, UK) were activated by immersion in methanol for 20 seconds and then washed in PBS for five minutes. The membrane was placed into a vacuum manifold Dot Blotter (Bio-Rad Laboratories, Watford, UK). Crude extracts from cells or culture supernatants were transferred onto the membrane by applying a vacuum and washed through with PBS. Without allowing the membrane to dry out, the Dot Blotter was dismantled and the membrane removed.

(b) Western Blotting

Samples of cell extracts and purified proteins were separated on SDS-PAGE as described in Section (xv). The Immobilon membrane was prepared for use as in (a) above. The gel and the membrane were assembled in the Semi-Dry Transfer Cell (Trans-Blot SD, Bio-Rad Laboratories) with the Immobilon membrane towards the anode and the SDS-PAGE gel on the cathode side. Between the cathode and the gel were placed 3 sheets of Whatman 3M filter paper cut to the size of the gel pre-soaked in a solution of 192 mM 6-amino-n-caproic acid, 25 mM Tris pH 9.4 containing 10% (v/v) methanol. Between the anode and the membrane were placed two sheets of Whatman 3M filter paper cut to the size of the gel and soaked in 0.3M Tris pH 10.4 containing 10% (v/v) methanol next to the anode and on this was laid a further sheet of Whatman 3M filter paper pre-soaked in 25 mM Tris pH 10.4 containing 10% (v/v) methanol.

The whole-assembled gel assembly was constructed to ensure the exclusion of air pockets. The proteins were transferred from the SDS-PAGE to the Immobilon membrane by passing 200 mA current through the assembly for 30 minutes.

(c) Immunoprobing of Dot Blot and Western Membranes

The membranes were blocked by incubating the membrane for 1 h at room temperature in 50 ml of 10 mM phosphate buffer pH 7.4 containing 150 mM NaCl, 0.02% (w/v) Ficoll 400, 0.02% (w/v) polyvinylpyrolidine and 0.1% (w/v) bovine serum albumin (BSA). The appropriate primary antibody was diluted to its working concentration in antibody diluent, 20 mM sodium phosphate buffer pH 7.4 containing 0.3 M NaCl, 0.5% (v/v) Tween-80 and 1.0% (w/v) BSA. The membrane was incubated for 2 h at room temperature in 50 ml of this solution and subsequently washed three times for 2 minutes in washing buffer, 20 mM sodium phosphate pH 7.4 containing 0.3 M NaCl and 0.5% (v/v) Tween-B80. The membrane was then transferred to 50 ml of antibody diluent buffer containing a suitable dilution of the species specific antibody labelled with the appropriate label, e.g. biotin, horse radish peroxidase (HRP), for the development process chosen and incubated for 2 h at room temperature. The membrane was then washed in washing buffer as described above. Finally, the blot was developed according to the manufacturer's instructions.

The appropriate dilution of antibody for both the primary and secondary antibodies refers to the dilution that minimises unwanted background noise without affecting detection of the chosen antigen using the development system chosen. This dilution is determined empirically for each antibody.

(xvii) Gene Sequences

The sequence of wild-type human C3d is available on public databases under accession number K02765. Other published C3d sequences include mouse Mus musculus (K02782), rat Rattus norvegicus (X52477), guinea pig Cavia porcellus (M34054), rabbit Oryctolagus cuniculus (M32434), sheep Ovis aries (AF038130), chicken Gallus gallus (U16848), cobra Naja naja (L02365), lamprey Lampetra japonica (D10087), toad Xenopus laevis (U19253), carp Cyprinus carpio (AB016210), trout Oncorhynchus mykiss (L24433) and sea urchin Strongylocentrotus purpuratus (AFO25526).

Variant gene sequences for human and mouse C3d are given in WO99/35260. The sequence of all novel cat C3d sequences and variant DNA sequences encoding concatamers of the same polypeptide are described in the following examples and in the appendices.

### EXAMPLES

1. Cloning of C3d from non-human vertebrate tissue using degenerate primers

1.1 Primer Design.

The degenerate primers used to clone the cat C3d sequences were designed by alignment of existing C3 protein sequences from human, mouse, rat, and guinea pig. Regions of amino acid conservation within and flanking the C3d region, where low codon redundancy was prominent were selected by eye, and oligonucleotides for RT-PCR designed to incorporate redundant bases where necessary

| | | |
|---|---|---|
| SEQID1 | (FARM 1) | TGY GGR GAR CAG AAC ATG ATY GGC ATG |
| SEQID2 | (FARM 2) | CCG TAG TAT CTY ASN TCR TTG AGC CA |
| SEQID3 | (FARM 3) | GGA GTC TTC GAG GAG AAT GGG CC |
| SEQID4 | (FARM 4) | GTG TGT CWG GRR CRA AGC CRG TCA TCA |
| | | T |
| SEQID5 | (FARM 5) | GTR ATG CAG GAC TTC TTC ATY GAC CTG |
| SEQID6 | (FARM 6) | GGC TGT CAG GGA CAC GTC TTT CTC |
| SEQID7 | (FARM 7) | GCA AGG GAC CCC MGT GGC CCA GAT G |
| SEQID8 | (FARM 8) | GYC ACC ACC GAC AAK GTG CCT TG |

R = G/A, Y = C/T, W = A/T, S = G/C, K = G/T, M = A/C, N = A/C/G/T.

1.2 Reverse Transcription-PCR

Total RNA was purified from liver or other tissue samples of cat (Felis catus) by the acid-guanidinium thiocyanate-phenol chloroform extraction technique of Chomczynski and Sacchi (Anal. Biochem 162: 156-159 (1987)). RNA extracted from bovine liver (Bos taurus) was obtained commercially (Clontech) . Approximately 3 ug of RNA was used in the RT reaction using the reverse transcription system from Promega. Reverse transcription was primed with 40 pmol of anti-mRNA sense primer, (ie. any of the even-numbered primers).

In some cases a single round PCR was sufficient to generate a positive product, whereas on others nested PCR was necessary. For example an outer PCR with primers FARM 4 and 5 was followed by inner PCR with primers FAPM 6 & 7 and 3 & 8, thus covering the entire C3d region. PCR conditions were typically 95° C. 30 sec, 54° C. 30 sec, 72° C. 60 sec, ×35 cycles.

1.3 Subcloning and Sequencing of Novel C3d Clones from Cat

PCR products derived from cat (Felis catus) were subcloned into pUC57/T (MBI Fermentas) and a minimum of three clones covering any region of C3d were fully sequenced on both strands. Sequence contigs were assembled and aligned using the SeqMan module of the DNAStar software package.

1.4 Design of Variant Genes to Prevent Homologous Recombination

For each native sequence published or cloned de novo variant genes may be designed which encode the same amino acid sequence but which contain a large number of silent mutations. These sequences may be cloned in isolation or in tandem with the native sequence and are resistant to homologous recombination. These sequences allow expression of concatamers of C3d from DNA which would otherwise undergo homologous recombination. In addition when used in DNA immunization vectors, or in vectors derived from live organisms with the intention of raising antibodies to antigens cloned in tandem to the C3d, the variant genes are resistant to homologous recombination with the native C3d present in the genome of the host species.

CAT C3d Nucleotide Sequence (Seq ID No. 9)
ACCCCCTCGGGCTGTGGGGAGCAGAACATGATAGGAATGACACCACGGTCATTGCAG
TCCATTGCCTGGACCAAACGGAGCAGTGGGAGAAGTTCGGCCTGGAAAAGAGGCAGGA
TTCCTTGCAGCTCATCGAAAAGGGGTACTCCCAGCAGCTGGCCTTCAGACAAGAGAAC
TCGGCTTTTGCGGCCTTCCAGAACCGGAAAGCCAGCACCTGGCTGACAGCCTACGTGG
TCAAGGTCTTCTCTCTGGCAGCCAATCTCATTGCCATCAACTCCCAAGTCCTCTGCGG
GGCTGTCAAATGGCTGATCCTGGAGAAGCAGAAGCCTGACGGGGTCTTCCAGGAAGAT
GGGCCCGTAATTCATCAAGAAATGACTGGTGGCTTCCGGGAGAACGAGGAGAAGGATG
TCGCCCTCACAGCCTTTGTTCTCATTGCGCTGCAAGAGGCGAAAGAGTTTTGCAATGA
TCAGGTCAATAGCCTGGAGCGCAGCATCACTAAGGCCGGAGATTATATTGAATTCCAC
TATAGGAACCTGCGGAGACCATACTCTGTGGCCATTGCAGGCTACGCCCTGGCCCAGT
CGGGCAGGCTGGAGAGAGACCTCCTTGAAAAATTTCTGAGCACAGCCAAAGAGAGGAC
AAGGTGGGAGGAGCCTGGCAAGAAACTCTACAGCGTGGAGGCCACATCCTATGCCCTC
TTGGCCCTGCTGGTGCTCAAAGACTTTGACTTTGTACGCCCCGTCGCCAGCTGGCTCA
ATGAGCAGAGATACTACGGAGGGGGCTACGGCTCCACCCAGGCCACCTTCATGGTGTT
CCAAGCCTTGGCCCAATACCAGAAGGATGTCCCTGACCACAAGGACCTGAACCTGGAA
GTATCCATTGAACTGCCA
Cat C3d amino acid sequence (Seq ID No 10)
TPSGCGEQNMIGMTPTVIAVHCLDQTEQWEKFGLEKRQDSLQLIEKGYSQQLAFRQEN
SAFAAFQNRKASTWLTAYVVKVFSLAANLIAINSQVLCGAVKWLILEKQKPDGVFQED
GPVIHQEMTGGFRENEEKDVALTAFVLIALQEAKEFCNDQVNSLERSITKAGDYTEFH
YRNLRRPYSVAIAGYALAQSGRLERDLLEKFLSTAKERTRWEEPGKKLYSVEATSYAL
LALLVLKDFDFVRPVASWLNEQRYYGGGYGSTQATFMVFQALAQYQKDVPDHKDLNLE
VSIELP

1. An isolated C3d polypeptide comprising an amino acid sequence. wherein the sequence is SEQ ID NO: 10 or a fragment thereof.

2. The polypeptide according to claim 1 is a recombinant C3d polypeptide or a derivative thereof which retains immunostimulatory activity.

3. An isolated nucleic acid which encodes a C3d polypeptide according to claim 1.

4. The nucleic acid according to claim 3 comprising a sequence, wherein the sequence is SEQ ID NO: 9 or a fragment thereof.

5. A variant nucleic acid sequence for use in a veterinary vaccine encoding a naturally occurring cat C3d polypeptide or fragment thereof which retains immunostimulatory activity, which by virtue of third base redundancy and/or other variations permissible within an amino acid codon, is non-identical to the naturally occurring DNA sequence encoding that polypeptide, or fragment.

6. The variant nucleic acid sequence according to claim 5 which encodes a C3d polypeptide comprising an amino acid sequence, wherein the sequence is SEQ ID NO: 10 or a fragment thereof which retains immunostimulatory activity.

7. A linear concatamer-comprising at least two non identical nucleic acid sequences which by virtue of third base redundancy and/or each other variations permissible within an amino acid codon each encode the same polypeptide according to claim 1.

8. A construct comprising a nucleic acid according to claim 4 or a variant thereof fused in frame to one or more nucleic acid sequences encoding an antigen.

9. A DNA immunization vector comprising a construct according to claim 8.

10. A veterinary composition comprising a nucleic acid according to claim 4 or a variant thereof and a physiologically acceptable excipient, carrier, or diluent.

11. A method for inducing an immune response to an antigen in a non human animal comprising administering to the animal an effective amount of the veterinary composition according to claim 10.

12. A method for manufacturing a medicament for inducing an immune response to an antigen in a non human animal comprising using a nucleic acid according to claim 4 or a variant thereof, wherein the nucleic acid is in the form of a linear concatamer, a construct, a DNA vector, or a veterinarv composition.

13. A construct comprising a linear concatamer according to claim 7 fused to one or more sequences encoding an antigen.

14. A DNA immunization vector .comprising a construct according to claim 13.

15. A veterinary composition comprising a nucleic acid according to claim 4 or a variant thereof, wherein the nucleic acid is in the form of a linear concatamer, a construct, or a DNA vector, and a physiologically acceptable excipient, carrier, or diluent.

16. A vector comprising a nucleic acid according to claim 4 or a variant thereof, wherein the nucleic acid is in the form of a linear concatamer or a construct according to claim 8.

17. An expression vector encoding a cat C3d polypeptide according to claim 1. wherein the vector whIgh is capable of directing expression of the C3d polypeptide in a prokaryotic or eukaryotic expression system.

18. A host cell comprising a vector according to claim 16.

19. An oligomeric protein comprising at least two, identical C3d polypeptides, each polypeptide comprising a sequence according to claim 1.

20. The oligomeric protein according to claim 19, wherein the protein is fused to an antigen.

21. A recombinant live organism comprising an oligomeric protein according to claim 19.

22. A veterinary composition comprising a fusion protein according to claim 19, and a physiologically acceptable carrier, excipient or diluent.

23. A method for inducing an immune response to an antigen in a cat comprising administering an effective amount of the veterinary composition according to claim 22 to the cat.

24. A method for manufacturing a medicament for inducing an immune response to an antigen in a cat comprising using an oligomeric protein according to claim 19, wherein the oligomeric protein is in a fusion protein or a veterinary composition.


SEQUENCE LISTING (10 sequences)

SEQ ID NO: 1 - DNA, 27 bp, Artificial Sequence
  Description of Artificial Sequence Synthetic primer
tgyggrgarcagaacatgatyggcatg

SEQ ID NO: 2 - DNA, 26 bp, Artificial Sequence
  Description of Artificial Sequence Synthetic primer
ccgtagtatctyasntcrttgagcca

SEQ ID NO: 3 - DNA, 23 bp, Artificial Sequence
  Description of Artificial Sequence Synthetic primer
ggagtcttcgaggagaatgggcc

SEQ ID NO: 4 - DNA, 28 bp, Artificial Sequence
  Description of Artificial Sequence Synthetic primer
gtgtgtcwggrrcraagccrgtcatcat

SEQ ID NO: 5 - DNA, 27 bp, Artificial Sequence
  Description of Artificial Sequence Synthetic primer
gtratgcaggacttcttcatygacctg

SEQ ID NO: 6 - DNA, 24 bp, Artificial Sequence
  Description of Artificial Sequence Synthetic primer
ggctgtcagggacacgtctttctc

SEQ ID NO: 7 - DNA, 25 bp, Artificial Sequence
  Description of Artificial Sequence Synthetic primer
gcaagggaccccmgtggcccagatg

SEQ ID NO: 8 - DNA, 23 bp, Artificial Sequence
  Description of Artificial Sequence Synthetic primer
gycaccaccgacaakgtgccttg

SEQ ID NO: 9 - DNA, 888 bp, Felis catus
accccctcgggctgtggggagcagaacatgataggaatgacacccacggtcattgcagtccattgcctggaccaaacggagcagtgggagaagttcggcctggaaaagaggcaggattccttgcagctcatcgaaaaggggtactcccagcagctggccttcagacaagagaactcggcttttgcggccttccagaaccggaaagccagcacctggctgacagcctacgtggtcaaggtcttctctctggcagccaatctcattgccatcaactcccaagtcctctgcggggctgtcaaatggctgatcctggagaagcagaagcctgacggggtcttccaggaagatgggcccgtaattcatcaagaaatgactggtggcttccgggagaacgaggagaaggatgtcgccctcacagcctttgttctcattgcgctgcaagaggcgaaagagttttgcaatgatcaggtcaatagcctggagcgcagcatcactaaggccggagattatattgaattccactataggaacctgcggagaccatactctgtggccattgcaggctacgccctggcccagtcgggcaggctggagagagacctccttgaaaaatttctgagcacagccaaagagaggacaaggtgggaggagcctggcaagaaactctacagcgtggaggccacatcctatgccctcttggccctgctggtgctcaaagactttgactttgtacgccccgtcgccagctggctcaatgagcagagatactacggagggggctacggctccacccaggccaccttcatggtgttccaagccttggcccaataccagaaggatgtccctgaccacaaggacctgaacctggaagtatccattgaactgcca

SEQ ID NO: 10 - protein, 296 aa, Felis catus
TPSGCGEQNMIGMTPTVIAVHCLDQTEQWEKFGLEKRQDSLQLIEKGYSQQLAFRQENSAFAAFQNRKASTWLTAYVVKVFSLAANLIAINSQVLCGAVKWLILEKQKPDGVFQEDGPVIHQEMTGGFRENEEKDVALTAFVLIALQEAKEFCNDQVNSLERSITKAGDYIEFHYRNLRRPYSVAIAGYALAQSGRLERDLLEKFLSTAKERTRWEEPGKKLYSVEATSYALLALLVLKDFDFVRPVASWLNEQRYYGGGYGSTQATFMVFQALAQYQKDVPDHKDLNLEVSIELP